(19) United States
(12) Patent Application Publication
(10) Pub. No.: US 2005/0070876 A1
(43) Pub. Date: Mar. 31, 2005

(54) Method and apparatus for administering a vaccine or other pharmaceutical

(75) Inventor: Thomas P. Castellano, Santa Monica, CA (US)

Correspondence Address: Pillsbury Winthrop LLP;Intellectual Property Group, 725 South Figueroa Street, Suite 2800, Los Angeles, CA, 90017-5406, US

(73) Assignee: PENJET CORPORATION, Santa Monica, CA (US)

(21) Appl. No.: 10/672,714

(22) Filed: Sep. 26, 2003

(51) Int. Cl. A61M 31/00

(52) U.S. Cl. 604/506; 604/173; 604/46

(57) ABSTRACT

An apparatus for administering a vaccine or other pharmaceutical includes a group of lancets that are exposed by the device to administer the injection. The group of lancets is housed within the device prior to actuation, and is recoiled into the device following the injection. The device may be actuated by depressing a trigger, and may include non-slip finger rests for ease of use. The device may additionally be prepackaged with the vaccine or other injectate. The device is a safe and convenient tool for administering vaccines and other pharmaceutical products; particularly those that require multiple, shallow skin piercings to effect proper delivery. The device of the present invention is particularly advantageous in the administration of a Smallpox vaccine. It is also designed for ease of manufacturing, as it may be assembled through an inline manufacturing process.

### FIELD OF THE INVENTION

This invention relates to the administration of vaccines and other pharmaceuticals, and, in particular embodiments, to vaccines and other pharmaceuticals that are administered into or below the layers of the skin.

### BACKGROUND OF THE INVENTION

The ability to properly administer vaccines and other pharmaceuticals is a critical component of modern healthcare. Children commonly receive an array of vaccinations, beginning at birth and continuing through adolescence. These inoculations generally include Hepatitis B (“Hep-B”), Diphtheria/Tetanus/Pertussis (“DTP”), Haemophilus influenzae type b (“Hib”), Polio (“IPV”), Pneumococcal conjugate (“PCV7”), Measles/Mumps/Rubella (“MMR”) and Varicella (or “Chickenpox”). Vaccinations for Hepatitis A (“Hep-A”), Influenza, Lyme Disease and/or Pneumococcal Polysaccharide may also be administered in particular instances. Moreover, because some vaccines have only a limited period of efficacy, they must be administered to individuals on a periodic basis to ensure resistance to infection; with certain vaccines, so-called “booster shots” are required. By way of example, the influenza vaccine, or “flu shot,” is routinely administered to adults on an annual basis at the beginning of the flu season in autumn. “Booster” Tetanus shots are recommended every ten years following the primary series of vaccinations that are administered during infancy, and upon an as-needed basis in response to cuts or puncture wounds in certain circumstances.

Most vaccines are administered by conventional needle injection. The depth of injection and corresponding configuration of the needle or needles required is different for various vaccines, yet the devices utilized (e.g., syringes, and in some instances, needle-less injectors) are relatively simple and may be easily operated by healthcare professionals. However, there are a host of vaccines and other pharmaceuticals (including many currently in the development pipeline at pharmaceutical companies around the world) whose conventional means of administration is significantly more complex, requiring skill, dexterity and careful attention to detail. By way of example, the Vaccinia (i.e., “Smallpox”) vaccine is among the vaccines in this category.

The Smallpox vaccine is generally administered into the skin over the insertion of the deltoid muscle or the posterior aspect of the arm over the triceps muscle. A multiple-puncture technique uses a pre-sterilized, bifurcated needle (such as those commercially available from Becton, Dickinson and Company; Franklin Lakes, N.J.) that is inserted vertically into a vaccine vial, causing a droplet of vaccine to adhere between the prongs of the needle. The droplet contains the recommended dosage of vaccine, and its presence within the prongs of the bifurcated needle is confirmed visually. Holding the bifurcated needle perpendicular to the skin, approximately fifteen punctures are rapidly made with strokes vigorous enough to allow a trace of blood to appear after 15-20 seconds. These punctures are made in an area no larger than 5 mm in diameter. Any vaccine remaining on the skin is wiped off with dry sterile gauze, and the gauze is disposed of in a biohazard waste container.

Some vaccines have never been administered to a large population, generally because they are still in an experimental phase (e.g., an HIV/AIDS vaccine). Others have not been administered for many years. For example, the Smallpox vaccine described above was last administered on a large-scale basis in the late 1970's. The disease was officially declared eradicated by the World Health Organization on May 8, 1980, and vaccination programs were terminated. Now, more than twenty years later, a significant portion of the world population has not been immunized, and may rapidly fall victim to a mass epidemic should an outbreak occur. Moreover, diseases such as Smallpox tend to spread quickly. Some of these diseases do not require skin-to-skin contact for infection. They can be airborne or be transmitted by other pathological means, such as by nosocomial transmission (i.e., the spread of a disease through a health care setting, such as a clinic or hospital, as is often the case with Ebola hemorrhagic fever (“Ebola HF”) in African health care facilities).

Particularly during a time when the potential for outbreak of infectious disease is high, there is a need in the art for a simple medical device to administer vaccines and other pharmaceuticals, such as the Smallpox vaccine, an AIDS/HIV vaccine or any number of other products. Such a device would provide healthcare professionals with an important tool to use in administering vaccinations and inoculations in a fast, safe and simple manner. Moreover, since non-healthcare professionals may also bear responsibility for vaccinating a population of individuals in an emergency situation, the need for a device that accomplishes these goals is now imperative.

### SUMMARY OF THE DISCLOSURE

In one embodiment of the present invention, a vaccine/pharmaceutical administration device includes a group of lancets encased in a housing. The group of lancets may be affixed to a lancet base that is biased between two springs: an injection spring and a safety spring. The injection spring provides the necessary force to drive the group of lancets out of the injection end of the device and into or through the skin of a vaccination recipient, while the safety spring subsequently recoils the group of lancets back into the housing following vaccine/pharmaceutical administration. Thus, during operation, the group of lancets is exposed from the interior of the device housing only during the vaccination or inoculation. However, in alternate embodiments of the invention, the group of lancets may be exposed from the interior of the housing to deposit a vaccine or other pharmaceutical on the sharp tips of the lancets. The device may be actuated by a pushbutton trigger that fractures a set of shear pins in mechanical communication with the lancet base. Non-slip finger rests may additionally be included on the housing to enhance stability and for further ease of use.

In another embodiment, the vaccination device may be prepackaged with a vaccine or other pharmaceutical, or a component of the final pharmacological product that incorporates a vaccine or pharmaceutical. Different vaccine and pharmaceutical packaging arrangements may be used in connection with various embodiments of the invention to achieve, for example, optimal drug storage conditions and to enable the use of a range of vaccine and other pharmaceutical formulations (e.g., multiple-component products, viscous formulations, etc.).

In a further embodiment of the present invention, a method of administering an injection or vaccine inoculation includes selecting an administration site, and deploying an injection with the aforementioned device. Prior to administering the vaccine or pharmaceutical product, a cap may be removed from the injection end of the device. The device may thereafter be actuated with the pushbutton trigger.

In a still further embodiment of the present invention, a method for manufacturing the aforementioned device includes an assembly process, which, in particular embodiments may include an inline manufacturing procedure. The housing is provided, and the safety spring, lancet base, injection spring and pushbutton trigger are placed into the housing. Shear pins may be included to mechanically stabilize the lancet base. A finger support may be secured to the housing; in various embodiments, by a rotational locking mechanism. Securing the finger support may compress the injection spring such that an application of force to the pushbutton trigger that breaks the shear pins releases the tension in the injection spring to drive the lancets out of the injection end of the housing. The safety spring thereafter provides sufficient force to recoil the lancets back into the housing.

### BRIEF DESCRIPTION OF THE DRAWINGS

FIG. 1a depicts a cross-sectional view of an injection device in accordance with an embodiment of the present invention. FIG. 1b depicts an elevational view of the injection device from the actuation end thereof, and FIG. 1c depicts an elevational view of the same injection device from the injection end thereof.

FIG. 2 depicts a partial cross-sectional view of an injection end of an injection device with a cap in accordance with an embodiment of the present invention. A soft matrix material is illustratively included between the cap and the group of lancets included in the device.

FIG. 3 depicts a cross-sectional view of a finger support with two finger rests in accordance with an embodiment of the present invention. The finger support illustratively depicted therein includes a locking channel.

FIG. 4a depicts a cross-sectional view of a housing in accordance with an embodiment of the present invention. The housing illustratively includes a series of grooves therein, as well as a locking tab. FIGS. 4b and 4c depict elevational views of the housing from the injection end and actuation end thereof, respectively.

FIG. 5a depicts an elevational view of a lancet base in accordance with an embodiment of the present invention. The lancet base illustratively includes a group of lancets. It further illustratively includes a series of guides configured about its axial circumference. FIGS. 5b and 5c depict elevational views of the lancet base from the injection end and actuation end thereof, respectively.

### DETAILED DESCRIPTION OF THE INVENTION

The present invention is based on a device that may be used to administer a vaccine or another pharmaceutical or injectate. It is particularly well suited for use with mammals in the treatment and prevention of human disease or in veterinary applications. The device may be particularly advantageous for the administration of vaccines and other pharmaceutical products that are delivered through the superficial epidermis; although, in alternate embodiments, the device may be used for the administration of vaccines and other pharmaceutical products at a variant delivery depth, such as intradermally, subdermally, subcutaneously or intramuscularly.

By way of example, Smallpox vaccine may be administered with the apparatus and methods of the present invention. Further, as it is believed that Smallpox vaccine may be effective in the treatment or prevention of diseases other than Smallpox (e.g., Monkeypox), references herein to “Smallpox vaccine” or “Smallpox vaccination” shall be understood as being applicable to the treatment and prevention of these other disease conditions, as well. However, Smallpox vaccine is merely one example of a product that is suitable for use in connection with the device and methods of the present invention.

Numerous other products may be used in alternate embodiments, including, but in no way limited to, HIV/AIDS vaccines, Hep-B vaccines, DTP vaccines, Hib vaccines, IPV vaccines, PCV7, MMR vaccines and Chickenpox vaccines. Vaccinations for Hep-A, Influenza, Lyme Disease and Tetanus (both the primary vaccination series and booster shots), as well as Pneumococcal Polysaccharide are still further examples of vaccines and other products that may be administered with various embodiments of the invention.

The device and methods of the present invention are uniquely well suited to the mass vaccination of a population in the event of an epidemic or biological warfare. Thus, by way of example, the Anthrax vaccine may be administered with the device and methods of the present invention. Anthrax vaccine is conventionally administered in a series of three primary, subcutaneous injections at 0, 2 and 4 weeks, and three booster vaccinations at 6, 12 and 18 months; the vaccine manufacturer (BioPort Corporation; Lansing, Mich.) recommends annual booster shots after the primary series and initial set of booster vaccinations. Intramuscular versions of this vaccine are presently being studied, which may also be suitable for use with the device of the present invention, as well as other formulations that may be developed for variant delivery depths. Additionally, an outbreak of tularemia may be addressed with the device and methods of the present invention. Tularemia may be spread through the bite of an infected insect or other arthropod (usually a tick or deerfly), by handling infected animal carcasses, by eating or drinking contaminated food or water, or by inhaling the bacterium Francisella tularensis. While no vaccine is presently available in the United States for tularemia, one is currently under review by the U.S. Food and Drug Administration. Other diseases commonly spread through bacterial infection, such as bacterial meningitis and Ebola HF, are still further examples of applications for the present invention. A vaccine for Ebola HF has yet to be developed, but the disease still represents a good example of the type of medical condition that may benefit from the device of the present invention; particularly, because the individuals responsible for performing a mass inoculation once a vaccine is developed are not likely to all be trained physicians.

In sum, as will be readily appreciated by one of skill in the art, there are a host of diseases and pathological conditions that may be addressed with the device and methods of the present invention, whether those diseases and conditions relate to a mass epidemic, to biological terrorism or by the more mundane transmission of illness in a human or animal population, and whether the vaccines and pharmaceutical products are presently available, under development or yet to be identified. In fact, the device and methods of the present invention may be used in connection with still further applications, such as veterinary practice (e.g., to vaccinate cattle).

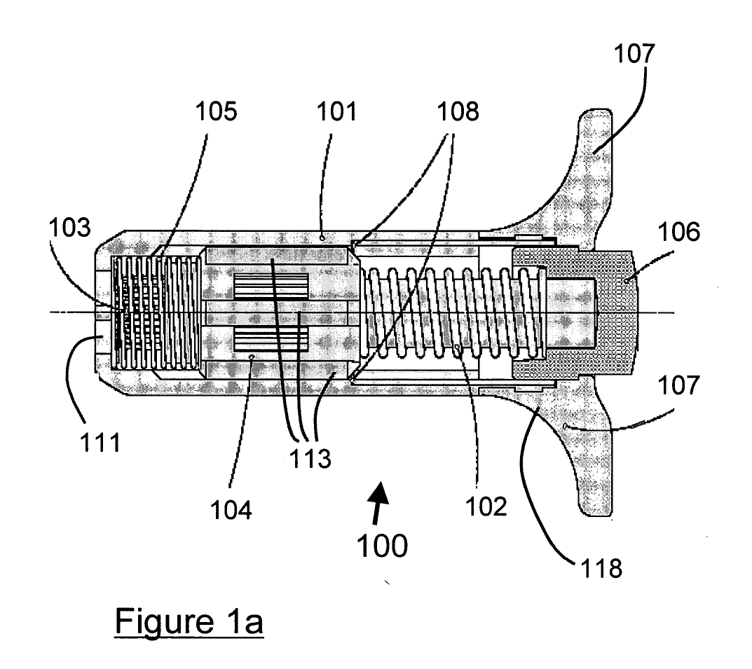

As illustratively depicted in FIG. 1, the device 100 includes a housing 101 with an injection end and an actuation end. The injection end may include an opening 111 through which a group of lancets 103 is exposed during the administration of a vaccination or injection of a pharmaceutical product. The group of lancets 103 may include any number of lancets (even just a single lancet), depending upon the particular delivery parameters of the vaccine or other pharmaceutical product being injected with the device 100. Further, the actuation end of the device 100 may include a pushbutton trigger 106 that a user may operate to actuate the device 100. The pushbutton trigger 106 may include a non-slip surface 117.

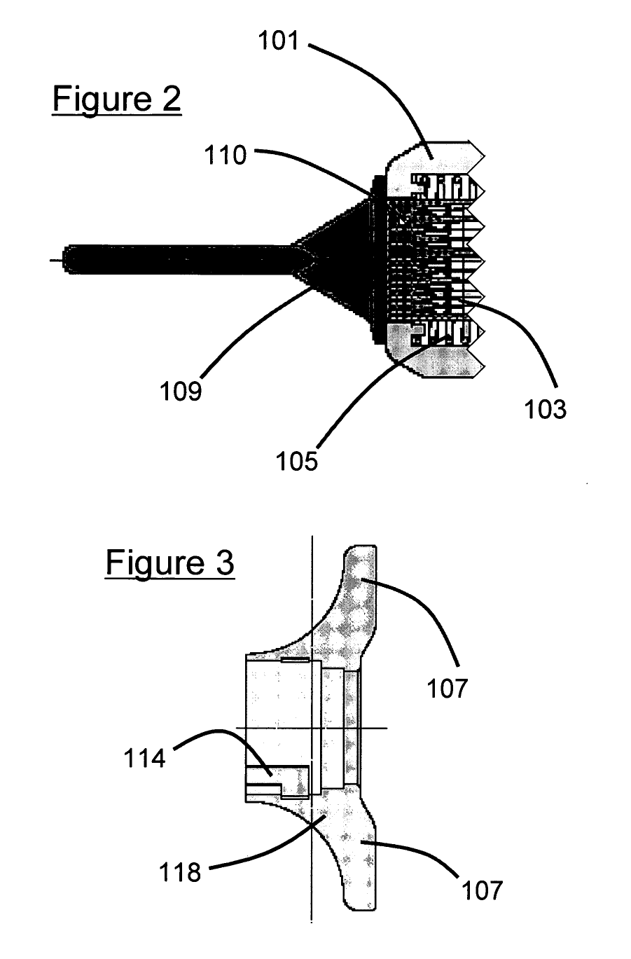

The housing 101 may also include finger rests 107 for ease of device operation. The finger rests 107 may be disposed upon a finger support 118 that is attached to the device housing 101. These finger rests 107 may include non-slip surfaces 116, and may impart device stability by providing a grip that readily conforms to the fingers of a user. They also provide a surface against which to squeeze in order for a user to create the force required against the pushbutton trigger 106 for device actuation. In the embodiment depicted in FIGS. 1 and 3, there are two such finger rests 107 disposed at 180° opposite one another along the exterior, axial length of the housing 101. Other configurations of finger rests 107 may be included in the device 100, as well.

The housing 101 may be configured to any suitable size and scale, depending on a variety of factors, such as, but in no way limited to, the delivery parameters of the vaccine or pharmaceutical product, manufacturing requirements for the device and the ease of operation and manual handling by a user. In embodiments of the device 100 configured for the administration of a Smallpox vaccine, the housing 101 may be less than about five inches in overall length; more preferably, less than about two inches in overall length. It will be readily understood, however, that housings of alternate lengths (for Smallpox vaccine or otherwise) remain within the ambit of the present invention, and may even be more desirable than the relatively short housing length described above in particular instances (e.g., to accommodate different gripping techniques employed by device users or to configure the devices for particular storage and shipping parameters).

The device 100 may further include a group of lancets 103, each having a sharp tip. The group of lancets 103 may be affixed to a lancet base 104 that is free to move axially within the device housing 101. In an embodiment of the present invention for the administration of Smallpox vaccine, there may be from about thirty to about forty lancets included in the group of lancets 103, although a variant number of lancets may be included in particular instances (e.g., to properly deliver a more or less concentrated version of a Smallpox vaccine that requires greater or fewer skin punctures, respectively, and to accommodate the particular delivery parameters of the wide variety of products that may be administered with the device 100). For example, in various embodiments of the invention, the group of lancets 103 may include one lancet, five lancets, ten lancets, twenty lancets or any number of lancets therebetween or greater. Any configuration of the group of lancets 103 in the lancet base 104 may be utilized. In one embodiment, the individual lancets are arranged approximately equidistant from one another throughout the surface area of the lancet base 104 nearest the injection end of the housing 101. Alternatively, the individual lancets may be arranged in a symmetrical pattern throughout the surface area of the lancet base 104 nearest the injection end of the housing 101. Other lancet arrangements may be used, as well. In one embodiment of the device 100 that is particularly well suited to administration of a Smallpox vaccine, there are approximately 36 lancets grouped approximately uniformly in the lancet base 104.

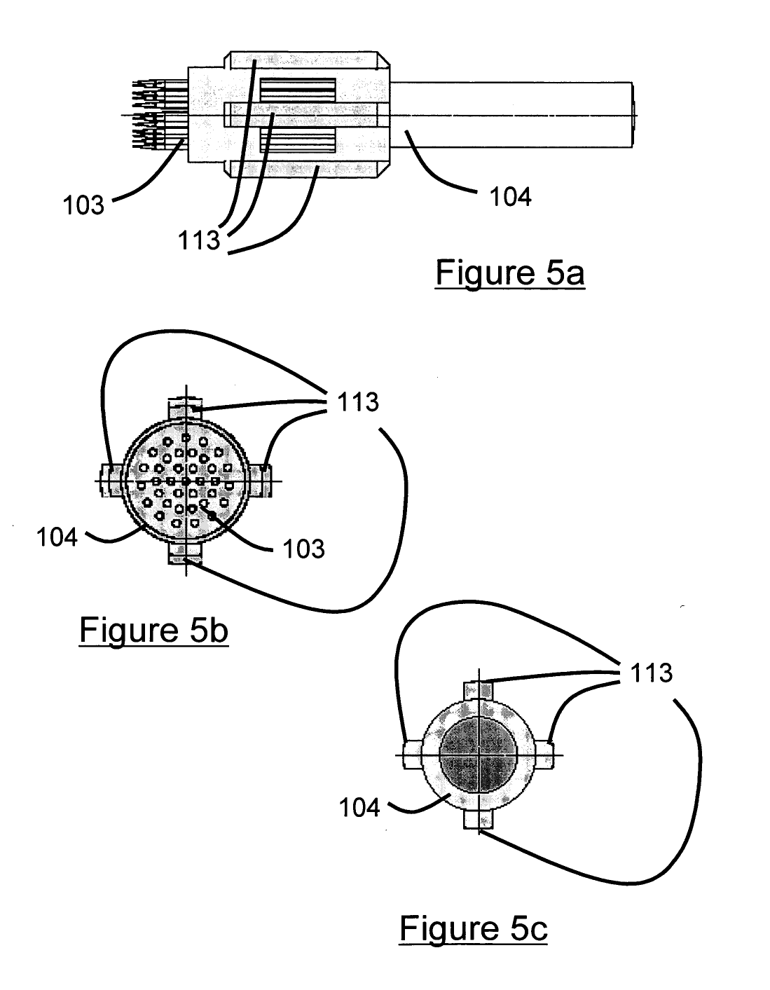

The lancet base 104 may additionally include guides 113 configured about its axial circumference. In one embodiment, these guides 113 communicate with corresponding grooves 112 in the interior surface of the housing 101. However, in alternate embodiments, no such grooves 112 are included in the housing 101. The guides 113 (whether or not corresponding grooves 112 are included) may provide axial stability during device use, while reducing the frictional force imparted by the internal axial surface of the housing 101 as the lancet base 104 slides through the housing 101. The lancet base 104 illustratively depicted in FIGS. 1 and 5 includes four guides 113 configured at 0°, 90°, 180° and 270° about the axis of the lancet base 104. Alternate guide configurations may be employed, as well. Additionally, the lancet base 104 may be in direct mechanical communication with the pushbutton trigger 106.

The lancet base 104 may be biased between two springs: an injection spring 102 and a safety spring 105. The injection spring 102 provides the necessary force to drive the group of lancets 103 out of the opening 111 in the injection end of the device 100 and force the sharp tips into and/or through the skin of a recipient. The safety spring 105 subsequently recoils the group of lancets 103 back into the housing 101. In this fashion, the group of lancets 103 is exposed from the interior of the housing 101 only during the administration of a vaccination or injection of another pharmaceutical product. In alternate embodiments, the device 100 may be configured such that the group of lancets 103 may be exposed from the interior of the housing 101 to deposit the vaccine on their sharp tips prior to device use. The injection spring 102 may be configured between the lancet base 104 and the pushbutton trigger 106; a portion of the lancet base 104 may reside within the injection spring 102. The safety spring 105 is configured between the lancet base 104 and the injection end of the housing 101; the group of lancets 103 and a portion of the lancet base 104 may reside within the safety spring 105.

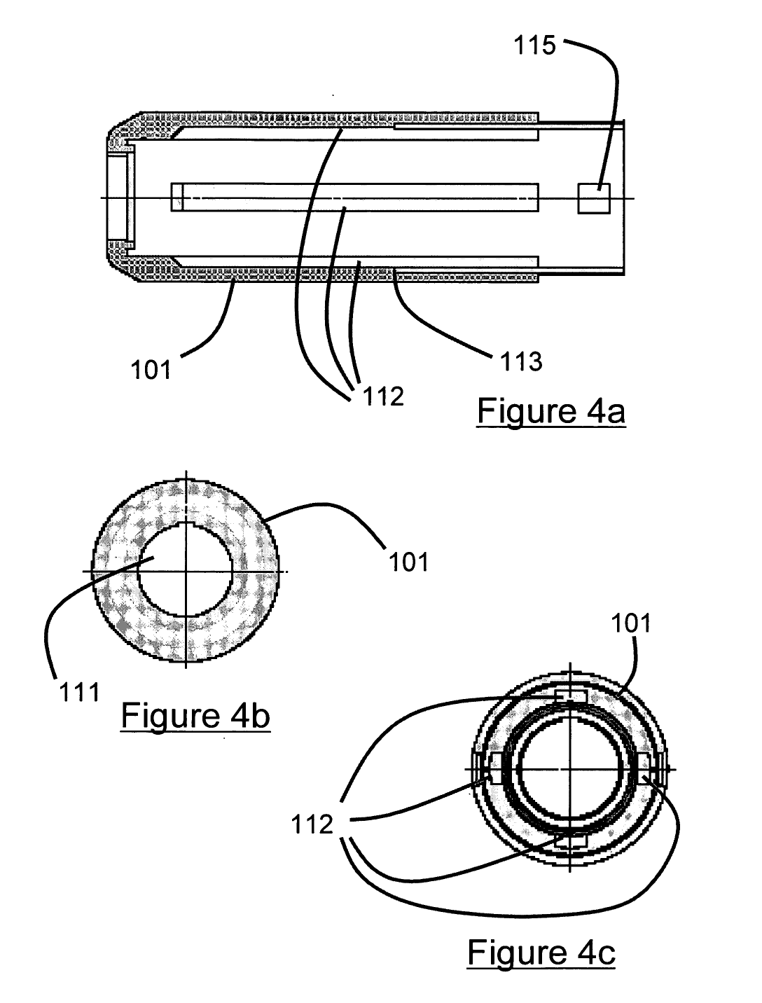

The lancet base 104 may be held in position with respect to the device housing 101 prior to actuation by shear pins 108. Any suitable number of shear pins 108 may be used in connection with various embodiments of the present invention, and they may be arranged in any desirable configuration, such as symmetrically about the longitudinal axis of the device 100. In one embodiment, two shear pins 108 are included and are disposed 180° opposite one another. The shear pins 108 are each attached on one end to the lancet base 104. On their opposing end, each shear pin 108 is in contact with the device housing 101. In one embodiment of the present invention, as depicted in FIG. 1, the ends of the shear pins 108 that contact the housing 101 rest against a ridge 113 (depicted in FIG. 4a) on the interior of the housing 101. When the device 100 is fully assembled, the force of the compressed injection spring 102 against the lancet base 104 forces the end of the shear pins 108 in contact with the device housing 101 against the ridge 113. Additionally, as depicted in FIG. 1, the lancet base 104 may be in direct mechanical contact with the pushbutton trigger 106. This may provide still further support for the shear pins 108 against the ridge 113. Upon application of sufficient force to the pushbutton trigger 106 the shear pins 108 fracture, thereby releasing the lancet base 104 such that it can move freely toward the injection end of the device 100. The compressed injection spring 102 may then drive the group of lancets 103 out of the opening 111 to administer the injection or vaccination.

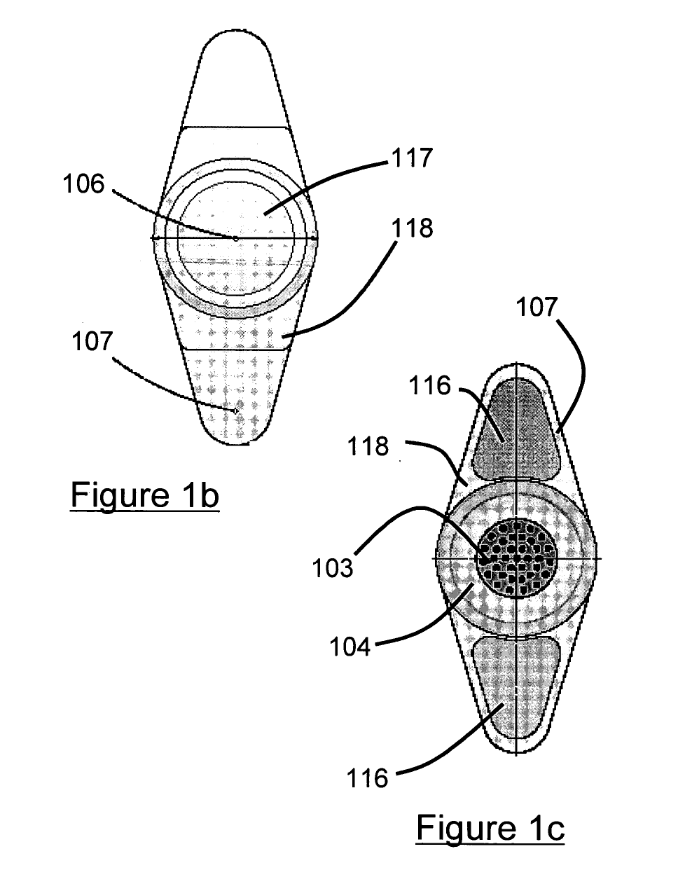

As illustratively depicted in FIG. 2, the device 100 may further include a cap 109, both in those embodiments where the device 100 is prepackaged with a vaccine or other pharmaceutical product, and those embodiments where the device 100 is not prepackaged with a vaccine or other pharmaceutical product. In embodiments where the device 100 is prepackaged with a vaccine or other pharmaceutical product, the sharp tips of the group of lancets 103 may be maintained in a soft matrix 110 that protects the sharp tips and prevents the product from leaking through the opening 111 configured on the injection end of the device 100. In embodiments where the device 100 is prepackaged with a viscous product (e.g., a paste) the sharp tips of the group of lancets 103 may be maintained in a casing that includes a well for each of the sharp tips (not shown). The wells may protect the tips and prevent the viscous product from escaping through the opening 111 configured on the injection end of the device 100. The cap 109 may be included around the soft matrix 110 or casing of wells (not shown), and may be removably attached to the injection end of the device housing 101.

As noted above, the device 100 may be prepackaged with a vaccine or other pharmaceutical, or a component of the final pharmacological product that incorporates a vaccine or pharmaceutical. In the latter instance, where a multiple-component vaccine or pharmaceutical product is utilized, a first component may be prepackaged with the device 100, while the second component may be maintained separately. The sharp tips of the group of lancets 103 including (e.g., coated with) the first component may then be “dipped” into a container with the second component; thereby generating a two-component pharmacological mixture for administration with the device 100. The sharp tips of the group of lancets 103 may be exposed from the opening 111 at the injection end of the device 100 to accomplish the dipping operation, although that is not required.

The device 100 may further be configured for inline manufacturing. The housing 101 may be provided, and the safety spring 105, lancet base 104 (with group of lancets 103 oriented toward the injection end of the housing 101), injection spring 102 and pushbutton trigger 106 may then be placed into the housing 101 (in the embodiment of the present invention illustratively depicted in FIG. 1, these components may be placed into the housing 101 in the aforementioned order). Shear pins 108 may be included to mechanically stabilize the lancet base 104, and may rest against a ridge 113 in the interior of the housing 101. A finger support 118 may then be secured to the housing 101; in various embodiments, by a rotational locking mechanism.

As illustratively depicted in FIGS. 3 and 4a, the rotational locking mechanism may include a locking tab 115 disposed on the interior of the housing 101 that communicates with a locking channel 114 configured on the interior of the finger support 118. The locking tab 115 may first slide through the locking channel 114 when the finger support 118 is placed on the actuation end of the housing 101. Once the finger support 118 is moved a predetermined length along the longitudinal axis of the housing 101, it may be rotated such that the locking tab 115 secures the finger support 118 to the housing. Securing the finger support 118 to the housing 101 may also compress the injection spring 102 between the pushbutton trigger 106 and lancet base 104, but not to such a great degree that the shear pins 108 break. This act of securing may also place the lancet base 104 and pushbutton trigger 106 in direct mechanical contact in such fashion as to support the shear pins 108 against the ridge 113. Any number of rotational locking mechanisms may be included in the device 100. In one embodiment, two such mechanisms are included (i.e., a pair of locking tabs 115 and locking channels 114 disposed 180° opposite one another. A cap 109 may be secured to the housing 101 at any point in the manufacturing operation (e.g., before placing the various components into the housing 101, after securing the rotational locking mechanism or at any other suitable point in the manufacturing operation). The cap 109 may protect the group of lancets 103 and/or coat the sharp tips of the group of lancets 103 with a vaccine or other pharmaceutical product.

### EXAMPLE

### Administration of a Vaccination

A device of the present invention is configured for inoculation with a Smallpox vaccine. An end cap is removed from the device and an appropriate vaccine administration site is selected (e.g., the skin over the insertion of the deltoid muscle or the posterior aspect of the arm over the triceps muscle). A group of 36 lancets is included in the device, and are pre-coated with a Smallpox vaccine. The injection end of the device is held against the surface of the skin at the vaccine administration site. The device is actuated by depressing a pushbutton trigger configured thereupon. Upon actuation, the group of 36 lancets coated with the Smallpox vaccine is exposed from an opening at the injection end of the device housing. These lancets pierce the skin of the recipient, thereby depositing the Smallpox vaccine into the recipient's superficial epidermis. The group of lancets is subsequently recoiled into the housing of the device by operation of a safety spring included therein. The device is then disposed of, and the vaccination is complete. Any excess vaccine remaining on the skin surface may be wiped off with sterile gauze.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

1. An injection apparatus, comprising:
a housing;
a group of lancets configured to move axially within the housing; and
an injection spring to drive the group of lancets out of the housing upon actuation of the injection apparatus.

2. The injection apparatus of claim 1, further including a pushbutton trigger in mechanical communication with the housing, wherein depression of the trigger actuates the injection apparatus.

3. The injection apparatus of claim 1, further including a lancet base to affix the group of lancets in a position relative to one another, wherein the lancet base is in mechanical communication with the injection spring.

4. The injection apparatus of claim 3, further including at least one shear pin in mechanical communication with both the lancet base and the housing, wherein the at least one shear pin maintains the mechanical position of the lancet base relative to the housing until the at least one shear pin is fractured upon device actuation.

5. The injection apparatus of claim 4, wherein the injection apparatus includes two shear pins disposed 180° opposite one another.

6. The injection apparatus of claim 3, further including a safety spring configured between the lancet base and an injection end of the housing, wherein the safety spring causes the lancet base to recoil axially within the housing after the group of lancets are at least partially exposed through an opening at the injection end of the housing.

7. The injection apparatus of claim 3, wherein the lancet base includes at least one guide to provide axial stability during administration of a vaccination.

8. The injection apparatus of claim 7, wherein the lancet base includes four guides configured at 0°, 90°, 180° and 270° about the lancet base.

9. The injection apparatus of claim 1, wherein the group of lancets includes from about thirty to about forty lancets.

10. The injection apparatus of claim 1, wherein the group of lancets includes thirty-six lancets.

11. The injection apparatus of claim 1, further including at least one finger rest.

12. The injection apparatus of claim 1, further including a pair of finger rests disposed opposite one another on the housing, each finger rest further including a non-slip surface.

13. The injection apparatus of claim 2, wherein the pushbutton trigger includes a non-slip surface.

14. The injection apparatus of claim 1, further including a cap removably attached to the housing.

15. The injection apparatus of claim 14, further including a soft matrix configured between the cap and the group of lancets.

16. The injection apparatus of claim 1, wherein the group of lancets are coated with a vaccine or other pharmaceutical product.

17. The injection apparatus of claim 16, wherein the vaccine or other pharmaceutical product is selected from the group consisting of a Vaccinia (“Smallpox”) vaccine, an Anthrax vaccine, a tularemia vaccine, an HIV/AIDS vaccine, a Hepatitis A (“Hep-A”) vaccine, a Hepatitis B (“Hep-B”) vaccine, a Diphtheria/Tetanus/Pertussis (“DTP”) vaccine, a Haemophilus influenzae type b (“Hib”) vaccine, a Polio vaccine (“IPV”), Pneumococcal conjugate (“PCV7”), a Measles/Mumps/Rubella (“MMR”) vaccine, a Varicella (“Chickenpox”) vaccine, an Influenza vaccine, a Tetanus vaccine, a Lyme Disease vaccine, Pneumococcal Polysaccharide, a vaccine booster shot and combinations of the same.

18. The injection apparatus of claim 16, wherein the vaccine or other pharmaceutical product is an Ebola hemorrhagic fever (“Ebola HF”) vaccine.

19. The injection apparatus of claim 17, wherein the vaccine or other pharmaceutical product is a Smallpox vaccine.

20. A method of injecting a vaccine or other pharmaceutical product, comprising:
selecting an injection site;
placing an injection apparatus against the injection site, the injection apparatus including:
a housing,
a group of lancets configured to move axially within the housing, the group of lancets including a vaccine or other pharmaceutical product, and
an injection spring to drive the group of lancets out of the housing upon actuation of the injection apparatus; and
actuating the injection apparatus to inject the vaccine or other pharmaceutical product.

21. The method of claim 20, further comprising removing a cap from the housing prior to placing the injection apparatus against the injection site.

22. The method of claim 20, wherein actuating the injection apparatus further includes depressing a pushbutton trigger on the injection apparatus.

23. A method of manufacturing an injection apparatus, comprising:
providing a housing;
introducing a safety spring into the housing to rest against an injection end of the housing;
introducing a lancet base into the housing and against the safety spring, the lancet base including a group of lancets, the group of lancets being oriented in the direction of the injection end of the housing;
introducing an injection spring into the housing and against the lancet base; and
mechanically coupling a pushbutton trigger to the housing.

24. The method of claim 23, wherein the lancet base includes at least one shear pin, the at least one shear pin coming to rest against a ridge in the housing upon mechanically coupling the pushbutton trigger to the housing.

25. The method of claim 23, wherein mechanically coupling the pushbutton trigger to the housing further includes securing a finger support to the housing by a rotational locking mechanism.

26. The method of claim 23, further including removably attaching a cap to the injection end of the housing.

27. The method of claim 26, wherein prior to removably attaching the cap to the injection end of the housing, the method further includes depositing a vaccine or other pharmaceutical upon the group of lancets.

28. The method of claim 26, wherein the cap includes a volume of vaccine or other pharmaceutical, and removably attaching the cap to the injection end of the housing deposits the volume of vaccine or other pharmaceutical upon the group of lancets.
